(19) United States
(12) Patent Application Publication
(10) Pub. No.: US 2026/0183199 A1
(43) Pub. Date: Jul. 2, 2026

(54) FLUORIDE-FREE DENTAL VARNISH COMPOSITION WITH NANO-HYDROXYAPATITE AND SLOW-RELEASE RESIN FOR ENHANCED ENAMEL REMINERALIZATION AND HYPERSENSITIVITY MANAGEMENT

(71) Applicant: Jennifer Eisenhuth, Mendota Heights, MN (US)

(72) Inventor: Jennifer Eisenhuth, Mendota Heights, MN (US)

(21) Appl. No.: 19/432,718

(22) Filed: Dec. 24, 2025

Related U.S. Application Data
(60) Provisional application No. 63/738,999, filed on Dec. 26, 2024.

(51) Int. Cl. A61K 6/20; A61K 6/838; A61K 6/887

(52) U.S. Cl. CPC A61K 6/20; A61K 6/838; A61K 6/887

(57) ABSTRACT

A dental varnish composition for promoting enamel remineralization may comprise nano-hydroxyapatite particles dispersed in a biocompatible resin matrix that forms an adhesive film on tooth enamel. The composition may include a solvent to facilitate uniform dispersion and one or more additives. The resin matrix may be hydrophilic to enhance adhesion and wetting of enamel microstructures. Contact with saliva triggers controlled, gradual release of nano-hydroxyapatite, providing sustained remineralization benefits and protection against demineralization over an extended period. The nano-hydroxyapatite may penetrate enamel microstructures and repair microlesions, while optionally reducing dentin hypersensitivity. The composition may be fluoride-free and formulated with defined weight ratios of resin, particles, and solvent. A method of treatment includes applying the varnish to tooth surfaces, forming the adhesive film, and allowing slow release of nano-hydroxyapatite to strengthen enamel.

### RELATED APPLICATIONS

This application claims the benefit of U.S. Provisional Patent Application No. 63/738,999, filed Dec. 26, 2024, titled “FLUORIDE-FREE DENTAL VARNISH COMPOSITION WITH NANO-HYDROXYAPATITE AND SLOW-RELEASE RESIN FOR ENHANCED ENAMEL REMINERALIZATION AND HYPERSENSITIVITY MANAGEMENT,” the entirety of the disclosure of which is hereby incorporated by this reference.

### TECHNICAL FIELD

The present disclosure relates generally to dental care compositions and, more particularly, to dental varnish formulations.

### BACKGROUND

Tooth enamel is susceptible to demineralization from routine dietary acids and biofilm activity. Exposure of dentin can lead to heightened sensitivity. Many existing coatings and oral care formulations are rapidly diluted and cleared by saliva and provide only short contact times at the tooth surface, resulting in limited effect and durability of effect under ordinary oral conditions.

In addition, stakeholders increasingly consider ingredient-exposure profiles and patient preferences when selecting products. The absence of sustained interaction with the tooth surface, together with incomplete penetration into enamel microstructures, can limit remineralization and the management of hypersensitivity, leaving room for improvement in both performance and user acceptance.

Historically, fluoride has been widely used in dental varnishes and oral care products due to its ability to enhance enamel resistance to acid attack and promote remineralization. Fluoride-based treatments have contributed significantly to reductions in caries prevalence over decades of use. However, concerns have emerged regarding potential overexposure, fluorosis, and patient preferences for fluoride-free alternatives. These considerations have driven interest in biocompatible, non-fluoride systems that can deliver comparable or superior benefits without associated drawbacks.

### SUMMARY

The present disclosure provides a clinically applied dental varnish that forms a persistent adhesive film on enamel, enabling sustained, localized release of nano-hydroxyapatite directly into enamel microstructures over an extended residence time.

According to some embodiments, the present disclosure relates to a dental varnish composition comprising: nano-hydroxyapatite particles; a resin that, when applied to tooth enamel, forms an adhesive film; a solvent that facilitates uniform dispersion of the nano-hydroxyapatite particles within the resin; and one or more additives; wherein the dental varnish composition is configured to provide controlled, extended (or slow) release of the nano-hydroxyapatite upon contact with saliva to promote enamel remineralization.

Particular embodiments may comprise one or more of the following features. The resin may comprise at least one of cellulose derivatives, polyvinylpyrrolidone (PVP), and bio-based gums. The nano-hydroxyapatite particles may have an average particle size of 20-100 nm. The composition may comprise 10% to 20% by weight of the nano-hydroxyapatite particles, 25% to 60% by weight of the resin, 10% to 30% by weight of the solvent, and up to 5% by weight of the one or more additives. The resin may be hydrophilic. The dental varnish composition may be fluoride-free.

According to some embodiments, the present disclosure relates to a dental varnish composition comprising a biocompatible resin matrix that, when applied to tooth enamel, forms an adhesive film, and nano-hydroxyapatite particles dispersed within the resin matrix, wherein the resin matrix is configured to gradually release the nano-hydroxyapatite to promote and facilitate enamel remineralization.

Particular embodiments may comprise one or more of the following features. The resin matrix may be hydrophilic and enhance wetting of enamel microstructures to improve adhesion and retention of the adhesive film. Contact with saliva may trigger gradual release of the nano-hydroxyapatite from the resin matrix. The gradual release may provide sustained remineralization benefits over an extended period while the adhesive film remains on the enamel. The resin matrix may function as a carrier and stabilizer that optimizes adherence of the composition to enamel and prolongs varnish efficacy. The composition may protect against enamel demineralization and strengthen enamel by facilitating mineral deposition. The nano-hydroxyapatite may penetrate enamel microstructures and repair enamel microlesions.

According to some embodiments, the present disclosure relates to a method of treating tooth enamel to promote remineralization, the method comprising applying to tooth surfaces a dental varnish composition comprising nano-hydroxyapatite dispersed in a biocompatible resin matrix, forming an adhesive film on the enamel, and allowing controlled, slow release of the nano-hydroxyapatite over an extended period.

Particular embodiments may comprise one or more of the following features. Contact with saliva may trigger the resin matrix to gradually release the nano-hydroxyapatite. The dental varnish composition may be fluoride-free. The dental varnish composition may comprise 10% to 20% by weight nano-hydroxyapatite having an average particle size of 20-100 nm. The method may further comprise reducing dentin hypersensitivity in a treated tooth. The nano-hydroxyapatite may repair enamel microlesions in the tooth surfaces. The gradual release may provide sustained benefits over an extended period while the adhesive film remains on the enamel.

The foregoing and other aspects, features, and advantages will be apparent from the DESCRIPTION and CLAIMS.

### DETAILED DESCRIPTION

The following detailed description provides numerous specific details. Those skilled in the relevant arts understand that embodiments of the disclosure may be practiced without these specific details. The disclosure may also be practiced in different and alternative configurations.

Unless specifically noted, it is intended that the words and phrases in the specification and the claims be given their plain, ordinary, and accustomed meaning to those of ordinary skill in the applicable arts. The singular forms “a,” “an,” and “the” include plural referents unless the context clearly dictates otherwise. Thus, for example, a reference to “a step” includes a reference to one or more of such steps. The words “exemplary,” “example,” “embodiment,” or various forms thereof are used herein to mean serving as an example, instance, or illustration. Any aspect or feature described herein as “exemplary” or as an “example” is not necessarily to be construed as preferred or advantageous over other aspects or designs. The examples are provided solely for purposes of clarity and understanding and do not limit or restrict the disclosure. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be understood that this disclosure is not limited to the specific materials, devices, methods, applications, conditions, or parameters described and herein, and that the terminology used herein is for the purpose of describing particular embodiments by way of example only and is not intended to be limiting of the claimed inventions. The term “plurality”, as used herein, means more than one.

As compositions, the embodiments described herein may be varied by routine formulation adjustments, including selection of polymer grade, solvent ratios, order of addition, and mixing conditions, without departing from the spirit of the disclosure.

Unless otherwise noted, percentages herein are by weight of the total composition.

### Definitions

As used herein, “nano-hydroxyapatite” (nHAp) refers to hydroxyapatite particles. In some embodiments, the nano-hydroxyapatite particles have an average particle size in the range of about 20-100 nm. In certain embodiments, the nano-hydroxyapatite particles have an average particle size in any of the following sub-ranges within 20-100 nm: about 20-60 nm, about 30-80 nm, or about 40-100 nm. In some embodiments, the average particle size is, for example, from about 10-150 nm or about 10-200 nm, provided the particles remain dispersible in the resin matrix and suitable for enamel contact under ordinary oral conditions.

As used herein, “sustained” or “gradual release” refers to release behavior that occurs over an extended period under ordinary oral conditions rather than as an immediate burst.

As used herein, “thin layer” refers to a film applied in a quantity sufficient to wet and cover enamel surfaces without excessive bulk.

As used herein, “oral environment” refers to ordinary oral conditions including contact with saliva, under which a composition may interact with enamel surfaces and microstructures.

As used herein, “treatment” refers to any application or process intended to prevent, reduce, or manage a condition affecting tooth enamel or dentin, including but not limited to promoting remineralization, reducing hypersensitivity, repairing microlesions, and protecting against demineralization. Treatment may include preventive, therapeutic, or maintenance interventions under ordinary oral conditions.

### Composition

The present disclosure is related to a dental varnish composition. In some embodiments, the dental varnish composition comprises nano-hydroxyapatite particles, a resin or resin matrix, a solvent, and/or one or more additives.

Nano-hydroxyapatite offers a biomimetic approach by closely resembling the mineral composition of natural enamel. Unlike fluoride, which primarily acts by forming a protective fluorapatite layer, nHAp can integrate directly into enamel microstructures, repairing microlesions and supporting natural mineral deposition. In some embodiments, nHAp may replace or supplement fluoride in preventive care strategies, providing sustained remineralization and hypersensitivity management without the risk of fluorosis or systemic exposure.

In some embodiments, the nano-hydroxyapatite particles are provided as ultra-fine, medical-grade nano-hydroxyapatite particles. In some embodiments, the nano-hydroxyapatite particles have an average size of 20-100 nm. The characteristics of the nano-hydroxyapatite may be selected to provide deep penetration into enamel microstructures for effective remineralization. In some embodiments, the nano-hydroxyapatite particles are present at about 10% to 20% by weight of the composition. In certain embodiments, nano-hydroxyapatite is present in sub-ranges within 10%-20% by weight, including about 10%-15%, about 12%-18%, or about 15%-20%. In some embodiments, the nano-hydroxyapatite may be present at broader weight percentages, for example from about 5% to about 30% by weight, or from about 2% to about 40% by weight, provided the composition maintains film integrity and suitable viscosity for thin-layer application. The nano-hydroxyapatite may include surface characteristics suitable for dispersion in hydrophilic matrices.

In some embodiments, the resin or resin matrix is a biocompatible resin system composed of natural or synthetic polymers. The resin matrix may be hydrophilic and may form a thin, adhesive film on teeth. In some embodiments, the resin matrix includes cellulose derivatives (e.g., hydroxyethyl cellulose, hydroxypropyl cellulose, hydroxypropyl methylcellulose, sodium carboxymethyl cellulose), polyvinylpyrrolidone (PVP), and bio-based gums such as chitosan. Additional hydrophilic film-formers may be used, alone or in combination, including polyvinyl alcohol (PVA), copovidone (PVP-VA), alginate, xanthan gum, gum arabic, pectin, hyaluronic acid, pullulan, and rosin/colophony-based varnish resins adapted for oral use. In some embodiments, the resin matrix is present at about 25% to 60% by weight of the composition. In certain embodiments, the resin matrix is present in sub-ranges within 25%-60% by weight, including about 25%-45%, about 30%-55%, or about 35%-50%. In other embodiments, the resin matrix content can be outside the foregoing range, for example from about 15% to about 75% by weight, or from about 20% to about 70% by weight, provided the matrix forms a continuous adhesive film on enamel.

In some embodiments, the solvent facilitates uniform dispersion of nano-hydroxyapatite and assists in varnish application and setting. The solvent system may include water and/or ethanol. One or more non-toxic stabilizing agents may be included to aid in maintaining homogeneity. Stabilizing/wetting agents may include lecithins, polysorbate-type materials, or poloxamers at levels sufficient to maintain dispersion and film uniformity. In some embodiments, the solvent system is present at about 10% to 30% by weight. In certain embodiments, the solvent system is present in sub-ranges within 10%-30% by weight, including about 10%-20%, about 12%-25%, or about 15%-30%. In some embodiments, the solvent system can include aqueous-alcohol mixtures and polyols (e.g., glycerol, propylene glycol) appropriate for oral use, and may be present at broader levels, for example from about 5% to about 40% by weight.

Optional additives may provide additional benefits. For example, xylitol can provide anticaries properties and enhanced enamel adherence, and essential oils (e.g., menthol, eucalyptus) can improve taste and provide slight antibacterial effects. Other optional additives may include taste modifiers (e.g., peppermint, spearmint, wintergreen), mild surfactants compatible with oral use, and flavor stabilization systems. In certain embodiments, optional additives are present up to 5% by weight, including subranges of up to about 1%, up to about 3%, or about 0.1%-5%. In some embodiments, total additives may be up to about 10% by weight, provided the composition retains film-forming performance.

In some embodiments, the varnish is fluoride-free. In certain contexts, fluoridefree formulations may address ingredient exposure preferences while supporting enamel health under ordinary oral conditions.

### Functional Attributes and Benefits

The nano-hydroxyapatite can mimic the natural composition of human enamel for facilitating effective remineralization and repair of microlesions. In some embodiments, the enamel-mimicking characteristics of nano-hydroxyapatite can support mineral deposition into existing microdefects. The disclosed composition can incorporate a high concentration of nano-hydroxyapatite within a controlled-release system to provide sustained benefits and serves as an alternative to fluoride dental varnishes.

The composition is formulated to adhere to enamel and form a thin, protective film that wets enamel microstructures. Film formation and wetting of enamel microstructures can increase contact time and uniformity of coverage. In some embodiments, the hydrophilic resin matrix functions as a carrier and stabilizer, maintaining dispersion of nano-hydroxyapatite while optimizing adhesion of the composition to enamel. By maintaining dispersion and adhesion, the resin matrix can facilitate sustained interaction with tooth surfaces, supporting consistent perceived outcomes. Under ordinary oral conditions, contact with saliva initiates gradual release of nano-hydroxyapatite from the resin matrix, providing extended interaction with the tooth surface.

In some embodiments, the adhesive film remains on enamel for a residence time of at least 30 minutes, at least 1 hour, at least 2 hours, or up to about 4 hours or more under ordinary oral conditions. During such residence time, the hydrophilic resin matrix can maintain wetting of enamel microstructures and sustained contact with the tooth surface, providing controlled, gradual release of nanohydroxyapatite for an extended period following application, which may be up to about 4 hours or more, thereby increasing the duration of interaction between nanohydroxyapatite and enamel microstructures.

Without intending to be bound by theory, such extended interaction provides prolonged efficacy and increased enamel contact, which can support mineral deposition within enamel microstructures and repair of microlesions. These effects can enhance enamel integrity and aid in the management of dentin hypersensitivity. In certain embodiments, the composition can help protect against demineralization while maintaining a benign sensory profile. In certain embodiments, extended interaction may be associated with improved management of sensitivity attributable to exposed dentin.

In certain embodiments, nano-hydroxyapatite can occlude dentinal tubules by depositing within or across tubule openings, thereby reducing fluid movement within the tubules under ordinary oral conditions. Without intending to be bound by theory, reduction in tubule fluid movement can be associated with decreased stimuli transmission and a corresponding reduction in dentin hypersensitivity.

Thus, the presently disclosed dental varnish composition offers a novel approach to dental enamel treatment, expanding options for preventive oral care with superior and sustained benefits.

### Methods of Preparation

Suitable preparation methods include dispersing the resin matrix in a selected solvent system, followed by incorporation of nano-hydroxyapatite under agitation sufficient to maintain dispersion. Mixing may be performed at ambient temperature using conventional equipment. In some embodiments, non-toxic stabilizing agents may be employed to maintain homogeneity. Optional additives (e.g., xylitol, essential oils) may be introduced during or after dispersion, as desired. The resulting varnish may be adjusted to a viscosity appropriate for thin-layer application. The order of addition may be varied, for example by preparing a resin pre-solution prior to addition of nano-hydroxyapatite, or by pre-wetting solids with a portion of the solvent system. Brief deaeration and/or filtration may be employed to reduce entrained air and aggregates and to support film uniformity, without altering the disclosed ranges.

### Methods of Use

In use, the varnish may be applied as a thin layer using an applicator or brush to enamel surfaces. Upon application, the resin matrix forms an adhesive film. When exposed to saliva, the matrix gradually releases nano-hydroxyapatite, allowing prolonged interaction and incorporation into enamel. In some embodiments, the composition may be provided in unit-dose or multi-dose packaging together with a soft applicator or brush configured for thin-layer application to enamel surfaces.

In some embodiments, following thin-layer application and film formation, the adhesive film persists on enamel for at least 30 minutes under ordinary oral conditions. In some embodiments, the adhesive film may persist for at least 1 hour or at least 2 hours or at least about 4 hours under ordinary oral conditions. Accordingly, controlled, gradual release of nano-hydroxyapatite may continue for a period that extends up to about 4 hours or more after application. In some embodiments, the method further comprises decreasing dentin hypersensitivity by occluding dentinal tubules with released nanohydroxyapatite, thereby reducing fluid movement within the tubules.

### EXAMPLES (PROPHETIC; NON-LIMITING)

Example 1 (PVP-based matrix): A varnish comprising about 15% nano-hydroxyapatite (20-100 nm), about 40% PVP resin matrix, about 20% ethanol: water solvent system, and about 2% optional additives including xylitol and a mint essential oil, with the balance being acceptable carriers and stabilizers. The composition is applied as a thin layer to form an adhesive film and exhibits gradual release under saliva exposure.

Example 2 (Cellulose derivative matrix): A varnish comprising about 12% nano-hydroxyapatite, about 35% hydroxyethyl cellulose matrix, about 25% water as primary solvent, and up to about 3% optional additives including xylitol. The composition is applied in a thin layer and demonstrates wetting of enamel microstructures and extended interaction under ordinary oral conditions.

Example 3 (Bio-based gum matrix): A varnish comprising about 18% nano-hydroxyapatite, about 50% chitosan matrix, about 15% ethanol: water solvent system, and up to about 5% optional additives including essential oils for taste; the film adheres to enamel and gradually releases nano-hydroxyapatite.

The disclosed compositions are not limited to the specific ingredients, order of addition, or processing conditions set forth herein. Ingredients performing similar functions may be substituted, and suppliers of equivalent materials may be used.

Percentages, ranges, and qualitative descriptors provided herein are by way of example and are inclusive and combinable unless stated otherwise. Values expressed with “about” include reasonable variations consistent with ordinary formulation tolerances.

Variations and modifications will occur to those of skill in the art after reviewing this disclosure. The features described may be implemented in any combination and sub-combination, and certain features may be omitted in particular embodiments without departing from the spirit of the disclosure.

Many additional implementations are possible. Further implementations are within the CLAIMS.

It will be understood that implementations of the preceding disclosure include but are not limited to the specific components disclosed herein, as virtually any components consistent with the intended operation may be utilized. Accordingly, for example, it should be understood that, while the text describes particular implementations, any such implementation may comprise any shape, size, style, type, model, version, class, grade, measurement, concentration, material, weight, quantity, and/or the like consistent with the intended operation.

In places where the description above refers to particular implementations, it should be readily apparent that a number of modifications may be made without departing from the spirit thereof and that these implementations may be applied to other implementations disclosed or undisclosed. The presently disclosed are, therefore, to be considered in all respects as illustrative and not restrictive.

We claim:

1. A dental varnish composition, comprising:
nano-hydroxyapatite particles;
a resin that, when applied to tooth enamel, forms an adhesive film;
a solvent that facilitates uniform dispersion of the nano-hydroxyapatite particles within the resin; and
one or more additives;
wherein the dental varnish composition is configured to provide controlled, slow release of the nano-hydroxyapatite upon contact with saliva to promote enamel remineralization.

2. The dental varnish composition of claim 1, wherein the resin comprises at least one of cellulose derivatives, polyvinylpyrrolidone (PVP), and bio-based gums.

3. The dental varnish composition of claim 1, wherein the nano-hydroxyapatite particles have an average particle size of 20-100 nm.

4. The dental varnish composition of claim 1, wherein the composition comprises 10% to 20% by weight of the nano-hydroxyapatite particles, 25% to 60% by weight of the resin, 10% to 30% by weight of the solvent, and up to 5% by weight of the one or more additives.

5. The dental varnish composition of claim 1, wherein the resin is hydrophilic.

6. The dental varnish composition of claim 1, wherein the dental varnish composition is fluoride-free.

7. A dental varnish composition, comprising:
a biocompatible resin matrix that, when applied to tooth enamel, forms an adhesive film; and
nano-hydroxyapatite particles dispersed within the resin matrix;
wherein the resin matrix is configured to gradually release the nano-hydroxyapatite to promote enamel remineralization.

8. The dental varnish composition of claim 7, wherein the resin matrix is hydrophilic and enhances wetting of enamel microstructures to improve adhesion and retention of the adhesive film.

9. The dental varnish composition of claim 7, wherein contact with saliva triggers gradual release of the nano-hydroxyapatite from the resin matrix.

10. The dental varnish composition of claim 7, wherein the gradual release provides sustained remineralization benefits over an extended period while the adhesive film remains on the enamel.

11. The dental varnish composition of claim 7, wherein the resin matrix functions as a carrier and stabilizer that optimizes adherence of the composition to enamel and prolongs varnish efficacy.

12. The dental varnish composition of claim 7, wherein the composition protects against enamel demineralization and strengthens enamel by facilitating mineral deposition.

13. The dental varnish composition of claim 7, wherein the nano-hydroxyapatite penetrates enamel microstructures and repairs enamel microlesions.

14. A method of treating tooth enamel to promote remineralization, the method comprising:
applying to tooth surfaces a dental varnish composition comprising nano-hydroxyapatite dispersed in a biocompatible resin matrix;
forming an adhesive film on the enamel; and
allowing controlled, slow release of the nano-hydroxyapatite over an extended period.

15. The method of claim 14, wherein contact with saliva triggers the resin matrix to gradually release the nano-hydroxyapatite.

16. The method of claim 14, wherein the dental varnish composition is fluoride-free.

17. The method of claim 14, wherein the dental varnish composition comprises 10% to 20% by weight nano-hydroxyapatite having an average particle size of 20-100 nm.

18. The method of claim 14, further comprising reducing dentin hypersensitivity in a treated tooth.

19. The method of claim 14, wherein the nano-hydroxyapatite repairs enamel microlesions in the tooth surfaces.

20. The method of claim 14, wherein the gradual release provides sustained benefits over an extended period while the adhesive film remains on the enamel.
